(19) United States
(12) Patent Application Publication
(10) Pub. No.: US 2024/0225976 A1
(43) Pub. Date: Jul. 11, 2024

(54) TRANSLUCENT COSMETIC COMPOSITION

(71) Applicant: Amorepacific Corporation, Seoul (KR)

(72) Inventors: Dongwan HA, Seoul (KR); Seunghan Park, Seoul (KR)

(73) Assignee: Amorepacific Corporation, Seoul (KR)

(21) Appl. No.: 18/477,748

(22) Filed: Sep. 29, 2023

(30) Foreign Application Priority Data
Jan. 10, 2023 (KR) 10-2023-0003604

(51) Int. Cl. A61K 8/42; A61K 8/04; A61K 8/55; A61Q 19/08

(52) U.S. Cl. CPC A61K 8/42; A61K 8/042; A61K 8/553; A61Q 19/08; A61K 2800/262

(57) ABSTRACT

Provided is a translucent cosmetic composition including: ceramide and/or a derivative thereof; a gelling agent; lecithin; and an oil.

### CROSS-REFERENCE TO RELATED APPLICATION

This application claims priority to and the benefit of Korean Patent Application No. 10-2023-0003604 filed in the Korean Intellectual Property Office on Jan. 10, 2023, the entire contents of which are incorporated herein by reference.

### BACKGROUND OF THE INVENTION

### (a) Field of the Invention

The present disclosure relates to a translucent cosmetic composition.

### (b) Description of the Related Art

Human bodies have a skin surface composed of keratinocytes that are matured and degenerated after differentiated from a basal layer. These keratinocytes are bound together by double-chain lamellar structures of ceramide, which is abundant in a stratum corneum and thus keeps the skin smooth and elastic.

The ceramide, which is a type of sphingolipid having a structure in which fatty acids are linked to sphingosine or phytosphingosine, accounts for about 40% or more of intercellular lipids constituting the stratum corneum and is an essential component for forming a structure of the stratum corneum or exhibiting a function thereof. This ceramide has been reported to have various physical functions in addition to properties as a major component of the stratum corneum.

The ceramide, when cells are damaged due to internal and external stress, removes the damaged cells to function to protect individuals from the stress. A content of the ceramide in the stratum corneum, as the skin ages, decreases, resulting in loss of skin moisture, exposure to external stimuli such as ultraviolet rays or chemicals, and exfoliation of the keratinocytes, which make the skin rough.

These symptoms may be improved by externally supplying the ceramide, for example, supplying it by cosmetics. However, when ceramides, which are sparingly soluble, are contained in a large amount in cosmetic formulations, there is a problem of deteriorating stability of the formulations, generating precipitation phenomena, and the like.

Accordingly, when the ceramides are included in the cosmetic formulations, most of them are in the form of hard capsules, resulting in generating foreign matters and thus bringing about a heavy feeling of use. The present inventors have made tireless efforts to develop a formulation type of cosmetic composition capable of providing a light feeling of use despite containing the ceramides, and finally completed the present invention.

### SUMMARY OF THE INVENTION

An embodiment provides a cosmetic composition of an emulsified gel cream formulation including ceramides having a light feeling of use.

According to an embodiment, a translucent cosmetic composition includes: ceramide and/or a derivative thereof; a gelling agent; lecithin; and an oil.

The ceramide derivative may include ceramide NP, ceramide NS, ceramide NH, ceramide AP, ceramide AS, ceramide EOP, ceramide EOS, or a combination thereof.

The ceramide and/or the derivative thereof may be included in an amount of about 0.5 wt % to about 1 wt % based on the total amount of the translucent cosmetic composition.

The gelling agent may be included in an amount of greater than or equal to about 1 wt % based on the total amount of the translucent cosmetic composition.

The gelling agent may be included in an amount equal to or greater than that of the ceramide and/or the derivative thereof.

The gelling agent may be included in an amount equal to or greater than that of the oil.

The gelling agent may include isostearyl alcohol*butylene glycol cocoate*ethyl cellulose.

The lecithin may be included in an amount equal to or less than that of the ceramide and/or the derivative thereof.

The lecithin may include hydrogenated lecithin.

The oil may be included in an amount of less than or equal to about 1 wt % based on the total amount of the translucent cosmetic composition.

The oil may include glyceride, diester, carbonate, caprylic/capric triglyceride, caprylic/capric/myristic/stearic triglyceride, dicaprylyl carbonate, diisostearyl maleate, ethylhexyl palmitate, ethylhexyl stearate, ethylhexyl cocoate, cetylethylhexanoate, octyldodecyl myristate, pentaerythrityl tetraethylhexanoate, jojoba oil, Queensland nut oil, olive oil, avocado oil, or a combination thereof.

The translucent cosmetic composition may be an emulsified gel cream formulation.

The cosmetic composition according to an embodiment can provide a light feeling of use without foreign matter despite containing ceramides.

### BRIEF DESCRIPTION OF THE DRAWINGS

FIG. 1 is a microscope photograph of a cosmetic composition according to Example 1.

FIG. 2 is a microscope photograph of a cosmetic composition according to Comparative Example 1.

FIG. 3 is a microscope photograph of a cosmetic composition according to Comparative Example 2.

FIG. 4 is microscope photograph of a cosmetic composition according to Comparative Example 3.

FIG. 5 shows photographs of when not coated and when each cosmetic composition according to Example 1 and Comparative Example 4 is coated to be 100 μm thick.

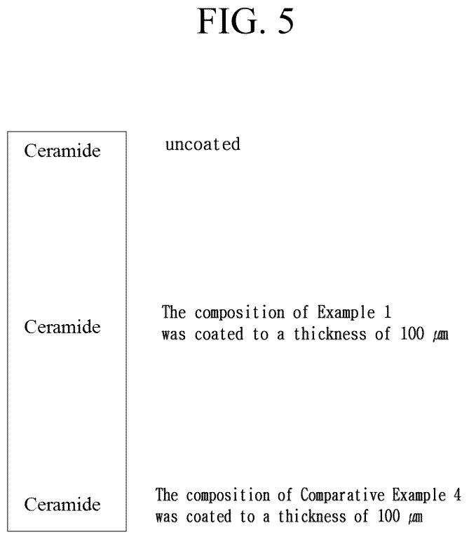

### DETAILED DESCRIPTION OF THE EMBODIMENTS

Hereinafter, example embodiments of the present invention will be described in detail. However, these example embodiments are only examples and do not limit the present invention. However, this disclosure may be embodied in many different forms and is not construed as limited to the example embodiments set forth herein.

In the present specification, unless explicitly described to the contrary, the word “comprise,” and variations such as “comprises” or “comprising”, will be understood to imply the inclusion of stated elements but not the exclusion of any other elements.

Hereinafter, a cosmetic composition according to an embodiment will be described.

A cosmetic composition according to an embodiment is a translucent cosmetic composition, including: ceramide and/or a derivative thereof; a gelling agent; lecithin; and an oil.

A term “translucent,” means a state in which, when an object is viewed through a medium, the object looks partially changed in terms of a shape and a color, and a term, “transparent,” means a state that the shape and the color of the object do not look changed but are visible, when viewed through the medium. The cosmetic composition according to an embodiment of the present invention may be expressed as a translucent cosmetic composition, which means that when an object is viewed through the cosmetic composition according to an embodiment, a shape and a color of the object look partially changed, and accordingly, the cosmetic composition according to an embodiment may not be interchangeably used with a transparent cosmetic composition. On the other hand, a term, “opaque,” means a state that the shape and the color of the object are not seen at all, when viewed through the medium, and accordingly, the cosmetic composition according to an embodiment may not be interchangeably used with the opaque cosmetic composition, just as it may not be interchangeably used with the transparent cosmetic composition.

Since a capsule including ceramide and/or a derivatives thereof is a hard formulation and thus has the biggest problem of feeling foreign matters during the use, recently, there have been attempts to formulate it into a gel type, for example, with viscosity of about 5000 cps or more, but in either case, a formulation technology of providing a light feeling of use without the foreign matters has not been known yet.

Accordingly, the present inventors conducted research on a formulation including the ceramide and/or the derivative thereof but also realizing the light feeling of use for several years, and eventually, completed the present invention after many trials and errors.

Conventionally, there have been attempts to remove the foreign matters by using a thickener such as carbomer, but the present inventors have succeed in formulating a composition into a translucent emulsified gel cream formulation by using the ceramide and/or the derivative thereof with a gelling agent, lecithin, and an oil, resultantly inventing a translucent cosmetic composition having the light feeling of use. When the cosmetic composition according to an embodiment is an opaque composition, there is a problem of a heavy feeling of use, but when the cosmetic composition is a transparent composition, the ceramide and/or the derivative thereof may be easily precipitated, or the foreign matters may be easily felt during the use, which shows that the cosmetic composition according to an embodiment should be formulated into the opaque composition.

When the cosmetic composition according to an embodiment is translucent, specifically, when the emulsified gel cream formulation-type translucent cosmetic composition according to an embodiment is evenly applied on white paper engraved with letters to a thickness of about 100 μm, the letters are clearly seen (some letters look neither crushed nor overlapped but clean), but when the emulsified gel cream formulation-type translucent cosmetic composition according to an embodiment is evenly applied on white paper engraved with letters to a thickness of about 200 μm, the letter are impossible or very difficult to identify, or even if identified, some letters may look crushed or overlapped.

The ceramide derivative may include, for example, ceramide NP, ceramide NS, ceramide NH, ceramide AP, ceramide AS, ceramide EOP, ceramide EOS, or a combination thereof, but the type is not necessarily limited thereto.

The ceramide and/or the derivative thereof may be included in an amount of about 0.5 wt % to about 1 wt % based on the total weight of the composition. For example, the ceramide and/or the derivative thereof may be included in an amount of about 0.5 wt % to about 1.5 wt %, about 0.5 wt % to about 1.4 wt %, about 0.5 wt % to about 1.3 wt %, about 0.5 wt % to about 1.2 wt %, about 0.5 wt % to about 1.1 wt %, about 0.5 wt % to about 1 wt %, about 0.5 wt % to about 0.9 wt %, about 0.5 wt % to about 0.8 wt %, about 0.5 wt % to about 0.7 wt %, or about 0.5 wt % to about 0.6 wt % based on the total weight of the translucent cosmetic composition according to an embodiment. At this time, when the content of ceramide and/or the derivative thereof is less than the above range, it is difficult to formulate in the form of a translucent emulsified gel cream, and when it exceeds the above range, it is difficult to stabilize the formulation.

However, when the ceramide and/or the derivative thereof is formulated into a gel cream formulation rather than a capsule formulation due to its characteristics, there is a problem of not easily securing stabilization even if a content thereof is controlled. In particular, in the emulsified gel-cream cosmetic composition containing the ceramide and/or the derivative thereof, since the stabilization of the ceramide and/or the derivative thereof has a significant influence on realization of the light feeling of use, it is very important to easily stabilize the ceramide and/or the derivative thereof in the emulsified gel cream formulation.

According to an embodiment, the stabilization of the ceramide and/or the derivative thereof may be easily achieved by using a gelling agent and an oil having an emulsification auxiliary function with the ceramide and/or a derivative thereof. Specifically, the stabilization of the ceramide and/or the derivative thereof in the emulsified gel cream formulation may be easily achieved by controlling types of the gelling agent, content ranges between the gelling agent and the oil, and the like.

For example, the gelling agent may include isostearyl alcohol*butylene glycol cocoate*ethyl cellulose. The isostearyl alcohol*butylene glycol cocoate*ethyl cellulose may not function as an active emulsifier but may work as a gelling agent assisting emulsification to some extent and thus, when used with the oil, may greatly contribute to the stabilization of the ceramide and/or the derivative thereof. When the gelling agent includes no isostearyl alcohol*butylene glycol cocoate*ethyl cellulose, the ceramide and/or the derivative thereof may be precipitated.

For example, the gelling agent may be included in an amount of about 1 wt % or more, based on the total amount of the translucent cosmetic composition. When the gelling agent may be included in an amount of less than about 1 wt % based on the total amount of the translucent cosmetic composition, the contribution to the stabilization of the ceramide and/or the derivative thereof may be deteriorated to a significant extent.

For example, the gelling agent may be included in an amount equal to or greater than that of the ceramide and/or the derivative thereof. For example, the gelling agent may be included at once to twice as much as the ceramide and/or the derivative thereof. When the contents of the gelling agent and the ceramide and/or the derivative thereof are controlled as aforementioned, the emulsified gel cream formulation including the ceramide and/or the derivative thereof may be easily stabilized.

For example, the gelling agent may be included in an amount equal to or greater than that of the oil. For example, the oil may be included in an amount of less than or equal to about 1 wt % based on the total amount of the translucent cosmetic composition. When the oil is included in an amount of greater than about 1 wt % based on the total amount of the translucent cosmetic composition according to an embodiment, the contribution to the stabilization of the ceramide and/or the derivative thereof may be deteriorated to a significant extent. In particular, when the content of the oil is greater than that of the gelling agent, there is a problem of hardly formulating it into a translucent emulsified gel cream.

For example, the oil may include glyceride, diester, carbonate, caprylic/capric triglyceride, caprylic/capric/myristic/stearic triglyceride, dicaprylyl carbonate, diisostearyl maleate, ethylhexyl palmitate, ethylhexyl stearate, ethylhexyl cocoate, cetylethylhexanoate, octyldodecyl myristate, pentaerythrityl tetraethylhexanoate, jojoba oil, Queensland nut oil, olive oil, avocado oil, or a combination thereof, but the type is not necessarily limited thereto.

For example, when the translucent cosmetic composition according to an embodiment does not include the oil, there occurs no precipitation of the ceramide and/or the derivative thereof, but since the ceramide and/or the derivative thereof is difficult to stabilize, the translucent cosmetic composition according to an embodiment should necessarily include the oil with the gelling agent together to stabilize the ceramide and/or the derivative thereof.

On the other hand, the translucent cosmetic composition according to an embodiment necessarily includes lecithin. When the lecithin is not included, like when the gelling agent includes no isostearyl alcohol*butylene glycol cocoate*ethyl cellulose, the ceramide and/or the derivative thereof may be precipitated.

For example, the lecithin may be included in an amount equal to or less than that of the ceramide and/or the derivative thereof. For example, the lecithin may be included in an amount of about 25 parts by weight to about 50 parts by weight based on 100 parts by weight of the ceramide and/or the derivative thereof. Herein, the lecithin, when included within the range, may suppress the precipitation of the ceramide and/or the derivative thereof and simultaneously help the stabilization of the emulsified gel cream formulation.

For example, the lecithin may include hydrogenated lecithin.

For example, the translucent cosmetic composition may be an emulsified gel cream formulation. In particular, the translucent cosmetic composition according to an embodiment has very low viscosity and thus may be used for a gel cream formulation-type composition rather than a toner, lotion, or essence formulation-type composition.

For example, the translucent cosmetic composition according to an embodiment may include components widely known and used by experts in the arts as conventional optional components for maintaining basic physical properties and quality, for example, oil-fat components, moisturizers, emollients, surfactants, organic or inorganic pigments, organic powders, ultraviolet (UV) absorbers, preservatives, bactericides, antioxidants, plant extracts, alcohols, pigments, fragrances, blood circulation promoters, cooling agents, antiperspirants, excipients, stabilizers, solubilizers, vitamins, and the like in addition to the above-described components.

Hereinafter, the present disclosure is illustrated in more detail with reference to examples. However, these are only examples, and the present disclosure is not limited thereto.

### Preparation of Cosmetic Composition in Gel Cream Formulation

According to each composition of Table 1 below, lipid-phased raw materials are heated and dissolved at 50° C. or higher, while being stirred, and aqueous-phased raw materials are heated and dispersed at 50° C. or higher, while being stirred. Subsequently, the lipid-phased materials dissolved in advance are added to the aqueous-phased materials and then emulsified for 3 minutes with a homogenizer and cooled, obtaining a cosmetic composition.

In Table 1 below, dipropylene glycol to isostearyl alcohol in order from top to bottom are mixed (dipropylene glycol part) and then heated and dissolved at 70° ° C. to 80° ° C. Separately, in Table 1 below, water and 1,2-hexandiol in order from top to bottom are mixed and then heated and dissolved at 70° C. to 80° C., and then the above dissolved dipropylene glycol part is added thereto and then emulsified, and subsequently, in Table 1, butylene glycol and hydroxyethyl acrylate/sodium acryloyldimethyl taurate copolymer*water/aqua/eau*sorbitan isostearate are added thereto and then emulsified again. When emulsified, homomixing is performed at 7000 rpm to 9000 rpm.

TABLE 1

| | | Comp. | Comp. | Comp. | Comp. |
|---|---|---|---|---|---|
| | Ex. 1 | Ex. 1 | E. 2 | Ex. 3 | Ex. 4 |
| DIPROPYLENE GLYCOL | 5 | 5 | 5 | 5 | 5 |
| CERAMIDE NP | 0.5 | 0.5 | 0.5 | 0.5 | 0.5 |
| HYDROGENATED LECITHIN | 0.25 | 0.25 | — | 0.25 | 0.25 |
| POLYGLYCERYL-6 | 0.25 | 0.25 | 0.25 | 0.25 | 0.25 |
| CAPRYLATE*POLYGLYCERYL-4 | | | | | |
| CAPRATE*WATER/AQUA/EAU | | | | | |
| CAPRYLIC/CAPRIC | 1 | 1 | 1 | — | 3 |
| TRIGLYCERIDE | | | | | |
| BUTYLENE GLYCOL | 3 | 3 | 3 | 3 | 3 |
| PALMITIC ACID*STEARIC ACID | 0.25 | 0.25 | 0.25 | 0.25 | 0.25 |
| ISOSTEARYL ALCOHOL | 1 | — | 1 | 1 | 1 |
| *BUTYLENE GLYCOL | | | | | |
| COCOATE*ETHYLCELLULOSE | | | | | |
| WATER/AQUA/EAU | to 100 | to 100 | to 100 | to 100 | to 100 |
| DISODIUM EDTA | 0.02 | 0.02 | 0.02 | 0.02 | 0.02 |
| GLYCERIN | 4 | 4 | 4 | 4 | 4 |
| SODIUM | 0.3 | 0.3 | 0.3 | 0.3 | 0.3 |
| POLYACRYLOYLDIMETHYL | | | | | |
| TAURATE | | | | | |
| ETHYLHEXYLGLYCERIN*TOCOPHEROL | 0.05 | 0.05 | 0.05 | 0.05 | 0.05 |
| GLYCERYL CAPRYLATE | 0.1 | 0.1 | 0.1 | 0.1 | 0.1 |
| TROMETHAMINE | 0.02 | 0.02 | 0.02 | 0.02 | 0.02 |
| 1,2-HEXANEDIOL | 1.5 | 1.5 | 1.5 | 1.5 | 1.5 |
| BUTYLENE GLYCOL | 3 | 3 | 3 | 3 | 3 |
| HYDROXYETHYL | 0.5 | 0.5 | 0.5 | 0.5 | 0.5 |
| ACRYLATE/SODIUM | | | | | |
| ACRYLOYLDIMETHYL TAURATE | | | | | |
| COPOLYMER*WATER/AQUA/EAU* | | | | | |
| SORBITAN ISOSTEARATE | | | | | |

### Evaluation 1: Confirmation of Translucency of Cosmetic Composition

The gel cream formulation cosmetic compositions according to Example 1 and Comparative Examples 1 to 4 are respectively coated to be 1 mm and 2 mm thick on white paper engraved with letters and then determined as transparent when the letters are clearly recognized at both of the thicknesses of 1 mm and 2 mm, as translucent, when the letters are clearly recognized at the thickness of 2 mm but not clearly recognized at the thickness of 1 mm, and as opaque when the letters are not clearly recognized at both of the thicknesses of 1 mm and 2 mm, and the results are shown in Table 2.

TABLE 2

| | Ex. 1 | Comp. Ex. 1 | Comp. Ex. 2 | Comp. Ex. 3 | Comp. Ex. 4 |
|---|---|---|---|---|---|
| Translucency | Translucent | Translucent | Translucent | Translucent | Opaque |

### Evaluation 2: Formulation Stability 1

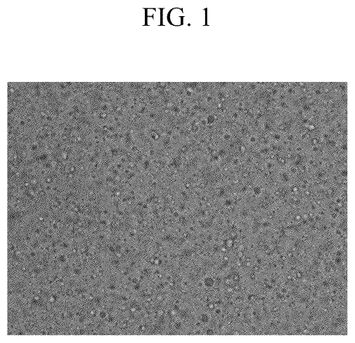

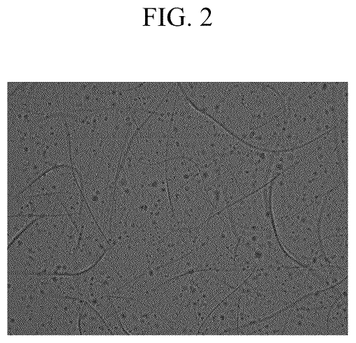

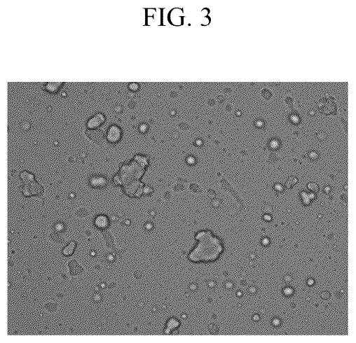

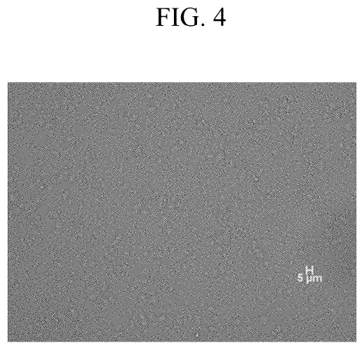

The gel cream formulation cosmetic compositions according to Example 1 and Comparative Examples 1 to 3, which are translucent cosmetic compositions, are photographed with a scanning electron microscope, and the results are shown in FIGS. 1 to 4.

Referring to FIGS. 1 to 4, the gel cream formulation cosmetic compositions according to Comparative Examples 1 and 2 exhibit precipitation of ceramide NP, and the gel cream formulation cosmetic composition of according to Comparative Example 3 does not exhibit the same precipitation in which the ceramide NP is uneven and thus forms no stable formulation. On the contrary, the gel cream formulation cosmetic composition according to Example 1 has no precipitation but exhibits very excellent formulation stability.

### Evaluation 3: Formulation Stability 2

The gel cream formulation cosmetic compositions according to Example 1 and Comparative Examples 1 to 3 are stored for 2 weeks under constant temperature conditions of 4° C., 25° C., 40° C., and 50° C., and then whether the formulations are aggregated or not is examined, and the results are shown in Table 3.

TABLE 3

| | Ex. 1 | Comp. Ex. 1 | Comp. Ex. 2 | Comp. Ex. 3 |
|---|---|---|---|---|
| 4° C. | stable | stable | stable | stable |
| Room | stable | unstable | unstable | stable |
| temperature | | (precipitation) | (precipitation) | |
| (25° C.) | | | | |
| 40° C. | stable | unstable | unstable | unstable |
| | | (precipitation) | (precipitation) | (precipitation) |
| 50° C. | stable | unstable | unstable | unstable |
| | | (precipitation) | (precipitation) | (precipitation) |

Referring to Table 3, in the compositions of the comparative examples, ceramide NP is precipitated and separated at room temperature or 40° C., and in the composition of Example 1, the precipitation does not occur at all from a low temperature of 4° C. to a high temperature of 50° C.

### Evaluation 4: Feeling of Use

The cosmetic compositions according to Example 1 and Comparative Examples 1 to 4 are scored with respect to feeling of use (light/normal/heavy) by selecting 30 Amorepacific in-house employees, and the scores are averaged and provided as results in Table 4.

TABLE 4

| | | Compar- | Compar- | Compar- | Compar- |
|---|---|---|---|---|---|
| | Example | ative | ative | ative | ative |
| | 1 | Example 1 | Example 2 | Example 3 | Example 4 |
| Feeling | light | normal | normal | normal | heavy |
| of | | | | | |
| use | | | | | |

Referring to Table 4, the translucent cosmetic composition according to an embodiment realizes a light feeling of use despite including ceramide and/or a derivative thereof.

Although the preferred embodiments of the present invention have been described in detail, the scope of the present invention is not limited thereto, and various modifications and improvements by those skilled in the art using the basic concept of the present invention defined in the following claims are also within the scope of the invention.

What is claimed is:

1. A translucent cosmetic composition, comprising
ceramide and/or a derivative thereof;
a gelling agent;
lecithin; and
an oil.

2. The translucent cosmetic composition of claim 1, wherein
the ceramide derivative includes ceramide NP, ceramide NS, ceramide NH, ceramide AP, ceramide AS, ceramide EOP, ceramide EOS, or a combination thereof.

3. The translucent cosmetic composition of claim 1, wherein
the ceramide and/or the derivative thereof is included in an amount of about 0.5 wt % to about 1 wt % based on the total amount of the translucent cosmetic composition.

4. The translucent cosmetic composition of claim 1, wherein
the gelling agent is included in an amount of greater than or equal to about 1 wt % based on the total amount of the translucent cosmetic composition.

5. The translucent cosmetic composition of claim 1, wherein
the gelling agent is included in an amount equal to or greater than that of the ceramide and/or the derivative thereof.

6. The translucent cosmetic composition of claim 1, wherein
the gelling agent is included in an amount equal to or greater than that of the oil.

7. The translucent cosmetic composition of claim 1, wherein
the gelling agent includes isostearyl alcohol*butylene glycol cocoate*ethyl cellulose.

8. The translucent cosmetic composition of claim 1, wherein
the lecithin is included in an amount equal to or less than that of the ceramide and/or the derivative thereof.

9. The translucent cosmetic composition of claim 1, wherein
the lecithin includes hydrogenated lecithin.

10. The translucent cosmetic composition of claim 1, wherein
the oil is included in an amount of less than or equal to about 1 wt % based on the total amount of the translucent cosmetic composition.

11. The translucent cosmetic composition of claim 1, wherein
the oil includes glyceride, diester, carbonate, caprylic/capric triglyceride, caprylic/capric/myristic/stearic triglyceride, dicaprylyl carbonate, diisostearyl maleate, ethylhexyl palmitate, ethylhexyl stearate, ethylhexyl cocoate, cetylethylhexanoate, octyldodecyl myristate, pentaerythrityl tetraethylhexanoate, jojoba oil, Queensland nut oil, olive oil, avocado oil, or a combination thereof.

12. The translucent cosmetic composition of claim 1, wherein
the translucent cosmetic composition is an emulsified gel cream formulation.
